(19) United States
(12) Patent Application Publication
(10) Pub. No.: US 2015/0157510 A1
(43) Pub. Date: Jun. 11, 2015

(54) OFFLOADING DEVICE AND METHODS OF USING SAME

(71) Applicant: Leslie Wald Smith, Plymouth, MN (US)

(72) Inventor: Leslie Wald Smith, Plymouth, MN (US)

(21) Appl. No.: 14/533,490

(22) Filed: Nov. 5, 2014

Related U.S. Application Data
(60) Provisional application No. 61/900,032, filed on Nov. 5, 2013.

(51) Int. Cl. A61F 13/06

(52) U.S. Cl. CPC A61F 13/069; A61F 2013/00272

(57) ABSTRACT

An offloading device that may be applied to the foot in order to redistribute weight and/or pressure around a wound and protect the wound from further damage. The offloading device, in some embodiments may be comprised of medical foam. The device may include one, two, three, or more layers of material. The shape of the device may be generally configured such that support and padding may be provided to an area around a wound, while the wound itself may not contact the material at all, or only marginally. The configuration of the device may include a supportive portion and a cutout portion. The device may be adhered to the foot by any suitable means, such as for example, adhesive. A removable backing may be attached to the adhesive layer, so that a user may peel the backing away prior to affixing the device to the foot.

### CROSS-REFERENCE TO RELATED APPLICATIONS

This application claims the benefit of U.S. Provisional Application No. 61/900,032, filed Nov. 5, 2013, the contents of all of which are hereby incorporated in their entirety by reference.

### FIELD OF THE INVENTION

The present invention relates to novel and advantageous offloading devices and methods of using the same. More particularly, the present invention in some embodiments is directed to a foam pad that may be adhered to or otherwise placed on or around the foot, or a portion of the foot to prevent injury, or further injury to a wound.

### BACKGROUND OF THE INVENTION

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

Wounds, and in particular, lower extremity wounds can result in significant medical and financial challenges to the healthcare system. This may be particularly true in the diabetic population where neuropathy can lead to ulceration, immobility, infection, and gangrene. Wound care protocols typically involve treating the underlying condition causing the wound, facilitating the wound healing process, and minimizing skin breakdown and wound recurrence. Offloading is important for reducing foot pressure points and for prevention as well as for healing. A typical offloading device is total contact casting (TCC). However, such devices may be cumbersome, expensive, unsightly and/or uncomfortable. Further TCC may lead to joint stiffness, muscle atrophy, labor-intensive application, and complications during cast removal. Other alternatives include having the patient endure bed-rest to avoid weight bearing, standard below-knee casts, and a charcot restraint orthotic walker. A number of additional options also exist, but there remains a need for an effective offloading device that is both comfortable to wear and cost-effective.

### BRIEF SUMMARY OF THE INVENTION

The following presents a simplified summary of one or more embodiments of the present disclosure in order to provide a basic understanding of such embodiments. This summary is not an extensive overview of all contemplated embodiments, and is intended to neither identify key or critical elements of all embodiments, nor delineate the scope of any or all embodiments.

The present disclosure, in one embodiment, relates to an offloading device having a first and second layer of medical grade foam. The first layer of medical grade foam may have a first side and a second side, and the second layer may be adhered to the first side of the first layer. The second side of the first layer may have an adhesive with a removable backing. The device may also have a third layer in some embodiments, which may also be composed of medical grade foam. In some embodiments, the first layer may have a cutout that may be placed over a wound. For example, the first layer may be configured in a U-shape, an O-shape, or an L-shape. The device may act to redistribute weight and/or pressure around a wound.

The present disclosure, in another embodiment, relates to a method for redistributing weight and/or pressure around a wound on a foot, the method including the steps of: providing an offloading device having at least a first layer of medical grade foam, wherein the first layer has a cutout for placement over a wound; and providing an attachment means such that the offloading device may be attached to the foot, allowing weight and/or pressure to be redistributed around the wound. The device provided as part of the method may further have at least a second layer of medical grade foam. The first layer of medical grade foam may be configured in a U-shape, an O-shape, or an L-shape. The attachment means provided as part of the method may be an adhesive with a removable backing in some embodiments.

While multiple embodiments are disclosed, still other embodiments of the present disclosure will become apparent to those skilled in the art from the following detailed description, which shows and describes illustrative embodiments of the invention. As will be realized, the various embodiments of the present disclosure are capable of modifications in various obvious aspects, all without departing from the spirit and scope of the present disclosure. Accordingly, the drawings and detailed description are to be regarded as illustrative in nature and not restrictive.

### BRIEF DESCRIPTION OF THE DRAWINGS

In the accompanying drawings, reference characters refer to the same parts throughout the different figures. The drawings are not necessarily to scale, emphasis has instead been placed upon illustrating the principles of embodiments of the present disclosure. With respect to the drawings:

FIG. 1 is a top view of an offloading device, in accordance with one embodiment.

FIG. 2 is a top view of an offloading device as applied to a foot, in accordance with one embodiment.

FIG. 3 is another view of the offloading device of FIG. 2.

### DETAILED DESCRIPTION

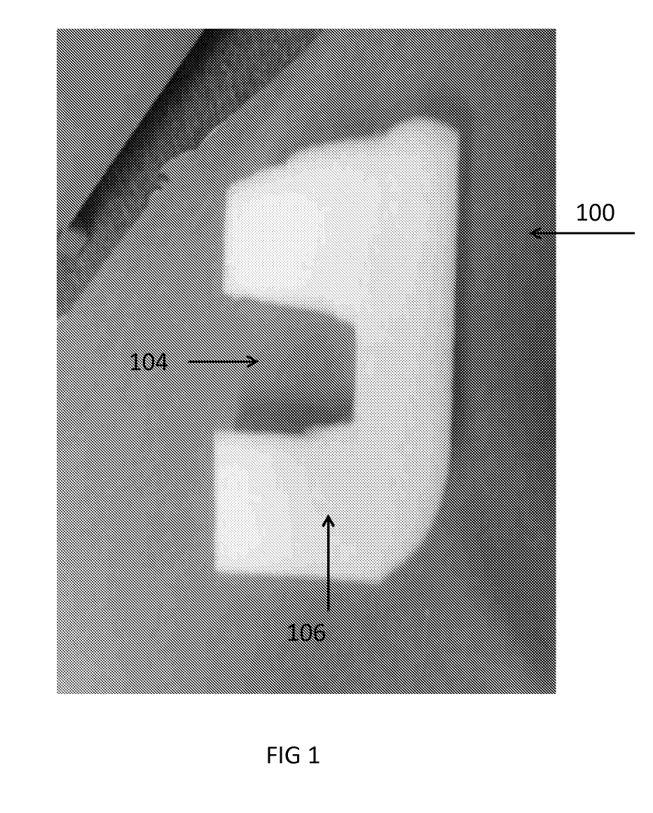

The present disclosure in some embodiments is directed to an offloading device that may be applied to the foot. FIG. 1 shows an embodiment of an offloading device 100 of the present disclosure. As may be seen, the offloading device, in some embodiments may be comprised of foam, medical foam, or any other relatively soft, pliable, yet supportive material, such as plastic, rubber, or any other suitable material. The device 100 may include one, two, three, or more layers of material. In embodiments that include more than one layer, the two or more layers may be comprised of the same material, or different materials. For example, at least one upper layer, to be placed against or near a user's skin, may be comprised of a soft foam, cotton, or other suitable and non-irritating material, while at least one lower layer may be comprised of a more supportive material such as plastic, rubber, a dense foam, or other suitable materials. Multiple layers may be coupled together by any suitable attachment means, such as for example, an adhesive material between the layers. Embodiments including more than one layer may have layers of the same shape, or may have layers of differing shapes. Layers may have various thicknesses, according to some embodiments. The shape of the device 100 may be generally configured such that support and padding may be provided to an area around a wound, while the wound itself may not contact the material at all, or only marginally. To this end, the configuration of the device 100 may include a supportive portion 106, comprising the one or more layers of material, to support the area surrounding the wound, and a cutout portion 104, such that when placed on the foot, the wound may not touch the device 100.

The supportive portion 106 of the device 100 may take any suitable and/or desirable shape. For example, the supportive portion 106 may be generally U-shaped, L-shaped, or O-shaped, for example to provide sufficient support to the area around the wound. FIG. 1 shows one example of a device with a U-shaped supportive portion 106. The supportive portion 106 may generally be any suitable size. In some embodiments, the size and/or shape of the supportive portion 106 may depend at least in part on the size of the wound and/or the size of the user's foot. The shape and/or size of the supportive portion 106 may, in some embodiments, depend at least in part on the location of the wound on the user's foot. For example, a wound located on a user's toe may require a different shape and/or size device 100 than a wound located on a user's heel.

The cutout portion 104 may be any desirable or suitable size and have any desirable or suitable shape to allow the wound to be free of contact from the device 100. The shape and/or size of the cutout portion 104 may generally depend at least in part on the shape and/or size of the wound. The cutout portion 104 may be entirely or partially surrounded by the supportive portion 106, according to some embodiments. Configuring the device 100 to allow the wound to remain uncovered may allow for topical wound care and/or placement and maintenance of biological skin substitute placement and protection, for example.

Generally, in some embodiments, the shape of the supportive portion 106 may be configured to help redistribute weight and/or pressure away from the wound. For example, where a wound is located at or near the walking surface of a user's foot, the device 100 may be placed about the wound so as to allow the user to apply weight to the foot without applying weight or as much weight to the wound itself. That is, the device 100 may redistribute a user's weight away from the wound location. In addition, redistribution of weight and/or pressure away from the wound may help contribute to improved local tissue oxygenation.

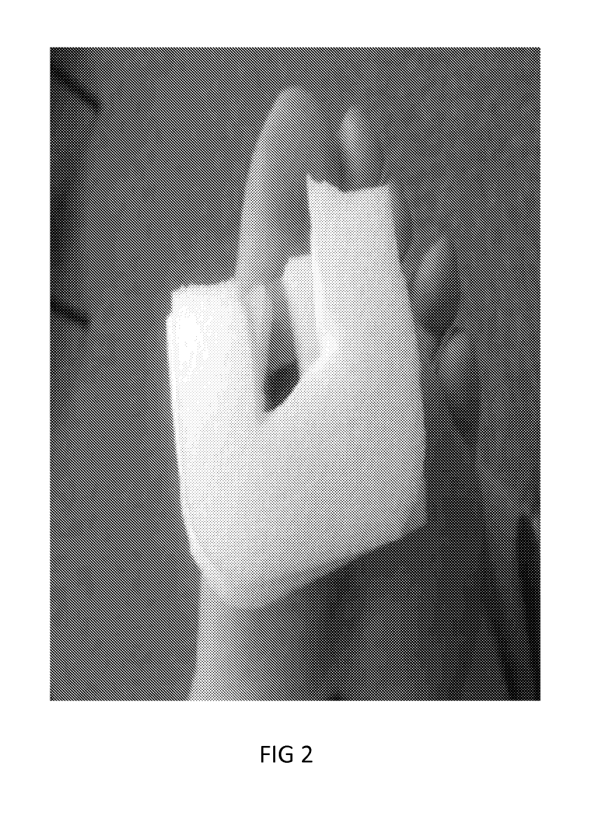

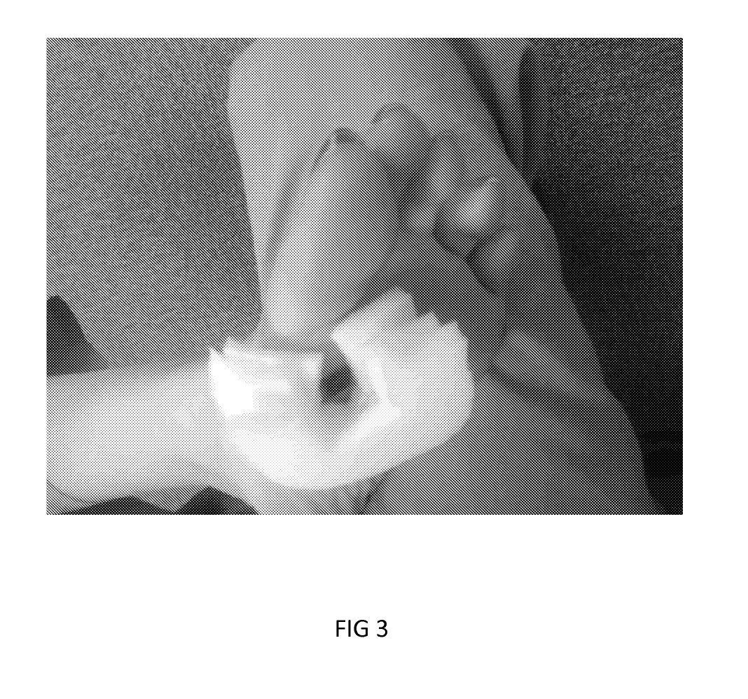

FIGS. 2 and 3 show the device 100 according to some embodiments of the present disclosure as it is positioned on the foot around a wound. The device 100 may be adhered to the foot by any suitable means, such as for example, adhesive. A removable backing may be attached to the adhesive layer, so that a user may peel the backing away prior to affixing the device 100 to the foot. As may be seen, the device 100 provides support and protection for the wound and the area around the wound, thus allowing its user to bear weight without risking further damage to the wound.

As used herein, the terms “substantially” or “generally” refer to the complete or nearly complete extent or degree of an action, characteristic, property, state, structure, item, or result. For example, an object that is “substantially” or “generally” enclosed would mean that the object is either completely enclosed or nearly completely enclosed. The exact allowable degree of deviation from absolute completeness may in some cases depend on the specific context. However, generally speaking, the nearness of completion will be so as to have generally the same overall result as if absolute and total completion were obtained. The use of “substantially” or “generally” is equally applicable when used in a negative connotation to refer to the complete or near complete lack of an action, characteristic, property, state, structure, item, or result. For example, an element, combination, embodiment, or composition that is “substantially free of” or “generally free of” an ingredient or element may still actually contain such item as long as there is generally no measurable effect thereof.

In the foregoing description various embodiments of the present disclosure have been presented for the purpose of illustration and description. They are not intended to be exhaustive or to limit the invention to the precise form disclosed. Obvious modifications or variations are possible in light of the above teachings. The various embodiments were chosen and described to provide the best illustration of the principals of the disclosure and their practical application, and to enable one of ordinary skill in the art to utilize the various embodiments with various modifications as are suited to the particular use contemplated. All such modifications and variations are within the scope of the present disclosure as determined by the appended claims when interpreted in accordance with the breadth they are fairly, legally, and equitably entitled.

What is claimed is:

1. An offloading device, comprising:
a first layer of medical grade foam, the first layer having a first side and a second side; and
a second layer of medical grade foam, adhered to the first side of the first layer;
wherein the second side of the first layer comprises an adhesive with a removable backing.

2. The offloading device of claim 1, further comprising a third layer adhered to the second layer.

3. The offloading device of claim 2, wherein the third layer comprises medical grade foam.

4. The offloading device of claim 1, wherein the first layer comprises a cutout that may be placed over a wound.

5. The offloading device of claim 1, wherein the first layer is configured in a U-shape.

6. The offloading device of claim 1, wherein the first layer is configured in an O-shape.

7. The offloading device of claim 1, wherein the first layer is configured in an L-shape.

8. The offloading device of claim 1, wherein the device acts to redistribute weight and/or pressure around a wound.

9. A method for redistributing weight and/or pressure around a wound on a foot, the method comprising the steps of:
providing an offloading device, the device comprising at least a first layer of medical grade foam, wherein the first layer comprises a cutout for placement over the wound;
providing an attachment means such that the offloading device may be attached to the foot, allowing weight and/or pressure to be redistributed around the wound.

10. The method of claim 7, wherein the offloading device further comprises at least a second layer of medical grade foam.

11. The method of claim 7, wherein the first layer of medical grade foam of the offloading device is configured in a U-shape.

12. The method of claim 7, wherein the first layer of medical grade foam of the offloading device is configured in an O-shape.

13. The method of claim 7, wherein the first layer of medical grade foam of the offloading device is configured in an L-shape.

14. The method of claim 7, wherein the attachment means comprises an adhesive with a removable backing.
